(19) United States
(12) Patent Application Publication
(10) Pub. No.: US 2006/0159735 A1
(43) Pub. Date: Jul. 20, 2006

(54) Method of reclaiming drug compound from liposomal drug manufacturing stream

(76) Inventors: Nicholas Bonham, Oxford (GB); Janet Poole, Oxford (GB); Andrew Wilkins, Wiltshire (GB)

Correspondence Address: Shu M. Lee;OSI Pharmaceuticals, Inc., Suite 110, 58 South Service Road, Melville, NY, 11747, US

(21) Appl. No.: 11/039,568

(22) Filed: Jan. 20, 2005

(51) Int. Cl. A61K 31/517; A61K 9/127

(52) U.S. Cl. 424/450; 514/266.2

(57) ABSTRACT

A method of forming a liposomal pharmaceutical composition containing the compound: 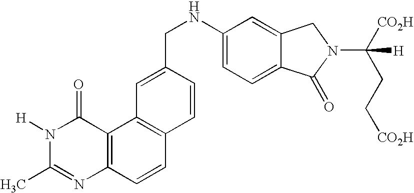 from a feedstock solution of the compound, comprises the steps of assaying a permeate wash for HSPC and cholesterol; processing the permeate wash to form a concentrated solution of the compound if the assay result is lower or equal to a predetermined value; and adding the concentrated solution to the feedstock solution.

### BACKGROUND OF THE INVENTION

The present invention is directed to a process to recover the drug compound from the manufacturing stream in the production of a liposomal drug product. In particular, the present invention is directed to a process to recover the OSI-7904 drug substance from the permeate washes that result from the manufacture of the liposomal drug product.

OSI-7904 is a benzoquinazoline compound represented by the chemical formula: 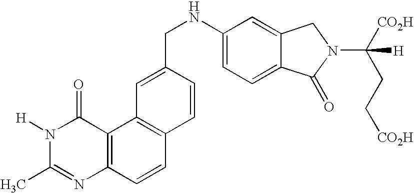
and is useful as a thymidylate synthase inhibitor having antitumour activity.

U.S. Pat. Nos. 6,306,865, 6,090,941, 5,663,337, 5,661,155 describe pharmaceutically active benzoquinazoline compounds. International Publication No. WO 01/95884 describes liposomal benzoquinazoline thymidylate synthase inhibitor formulations.

The process for preparing the liposomal OSI-7904 drug product has limited efficiency in terms of its usage of the drug compound. The amount of drug compound actually incorporated into the liposomes is considerably less than half. The remaining drug remains in the liquid outside the liposomes. Presently, this non-encapsulated drug compound is removed by an ultrafiltration process, involving a series of successive permeate washes. Practically all the non-encapsulated drug ends up in these permeate washes that contain ever decreasing amounts of the drug compound.

The successive permeate washes are then pooled (combined) and the combined liquid's pH is adjusted to 3 to precipitate the drug compound. The drug is then filtered off, washed with water and dried. The dry solid is assayed and purified as needed. Finally, the purified reclaimed drug compound is reused in the liposomal process.

This reclaiming process has several disadvantages. The solid drug compound is cytotoxic and must be treated as a hazardous material. The precipitation process must be performed at a facility able to process toxic solids—leading to the need to transport large volumes of permeate washes with the hazards and costs associated with such transportation. The biological and chemical stability of the drug might be problematic from such handling. Furthermore, it has been observed that the levels of two impurities:

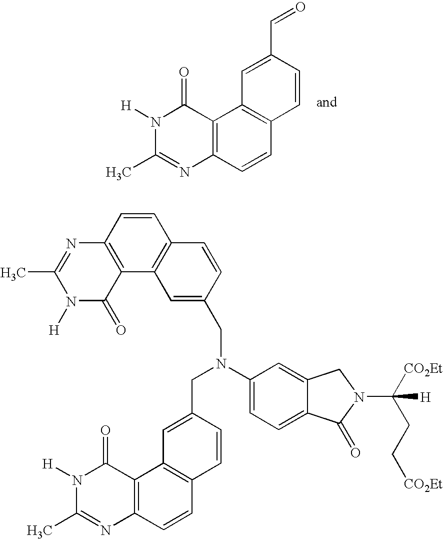

were found to change with such solid reclamation steps. This may be due to these impurities being incorporated into the liposome to a different extent from that of the drug compound.

Recycling by precipitation has severe disadvantages. Precipitation likely yields very fine suspensions that are slow to filter, difficult to wash, and leading to significant losses on handling. Precipitation of the solid and drying involves potential exposures to the highly toxic solid. Accordingly, barrier isolation technology is required to contain the toxic material below acceptable exposure levels. Finally, precipitation, filtration, and drying unit operations are less common in secondary manufacturing environments. Thus, it might be needed to transfer large amounts of wash solutions to a primary manufacturing site for recovery, which involves significant costs and risks.

Accordingly, it would be desirable to develop a reclamation process for OSI-7904 from the liquid outside the liposomes that does not involve drying the compound before reclamation. Such desirable reclamation would reduce risks to personnel because dust and powder are much harder to control than solutions, and would provide easier containment if the desirable reclamation involves a closed liquid system.

### SUMMARY OF THE INVENTION

The present invention is directed to a method of formation of a liposomal pharmaceutical composition containing the compound: 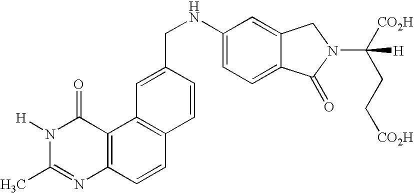
from a feedstock solution of the compound, comprising the steps of assaying a permeate wash for HSPC and cholesterol; processing the permeate wash to form a concentrated solution of the compound if the assay result is lower or equal to a predetermined value; and adding the concentrated solution to the feedstock solution.

### BRIEF DESCRIPTION OF THE DRAWINGS

FIG. 1 is a schematic flowchart diagram of an embodiment of the present invention.

FIG. 2 is a Bjerrum plot of the titration of 10 mL of 0.33 mM OSI-7904 aqueous solution in the presence of 5 mL chloroform.

### DETAILED DESCRIPTION OF THE INVENTION

The present invention is directed to a method of forming a liposomal pharmaceutical composition containing the compound: 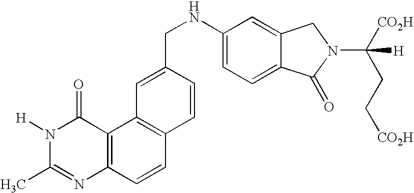
from a feedstock solution of the compound, comprising the steps of

assaying a permeate wash for HSPC and cholesterol;

processing the permeate wash to form a concentrated solution of the compound if the assay result is lower or equal to a predetermined value; and

adding the concentrated solution to the feedstock solution.

Further, the method includes extracting the permeate wash to form an extracted solution if the assay result is higher than a predetermined value. The method includes testing the extracted solution, to form a tested solution, to determine if the HSPC and cholesterol is higher than a predetermined value. The method also includes processing the tested solution to form a concentrated tested solution of the compound if the test results are not higher than a predetermined value. The method also includes adding the concentrated tested solution to the feedstock solution.

The manufacturing of a liposomal drug product such as that of OSI-7904 can be described in general terms as including the steps of:

1) Mixing the drug compound with a liquid carrier such as, for example water at an appropriate pH

2) Typically, multilamellar vesicles (MLV) are prepared by adding, for example, cholesterol and hydrogenated soy phophatidylcholine (HSPC) in a pre-prepared mixture. MLVs are liposomes consisting of alternating series of concentric membrane bilayers with the spaces in between the bilayers filled with aqueous solution.

3) In a homogenization step, the MLV/solution mixture is put through a high shear process such as sonication. This forms a dispersion of small unilamellar vesicles that are the desired liposomal drug product.

At this point, an ultrafiltration process can be used to isolate the desired liposomal drug product. Ultrafiltration involves placing the liposomal dispersion at one side of a semi-permeable membrane with clean wash water on the other side of the membrane. Substances in free solution equilibrate at the two sides of the membrane—thereby lowering the concentration of the dissolved drug compound in the liposomal dispersion liquid and raising the concentration in the permeate wash water. The permeate water is removed and fresh wash water is introduced as a successive wash step.

There is a decreasing concentration of free drug compound in the permeate water from each successive wash step. For example, the first wash might yield about 13 mg/mL concentration of OSI-7904, the second may yield about 7 mg/mL, and the third may yield about 4 mg/mL. Accordingly, ultrafiltration leads to a large amount of water containing bioactive compounds that must be treated with care.

Experiments were conducted to i) separate the OSI-7904 from the cholesterol and HSPC in the permeate washes and to ii) concentrate the OSI-7904 in aqueous solution directly.

Purification methods attempted to separate the OSI-7904 from the cholesterol and HSPC in the permeate washes included:

1) Liquid-liquid extraction. Extraction of the permeate with a hydrophobic solvent that is immiscible with water to remove the HSPC and cholesterol has certain attractions. Chloroform was chosen since it is used in the existing process so methods of handling and assaying are already available. Further, it would be possible to remove any chloroform remaining in the permeate by evaporation.

2) Solid Phase extraction. Passing the permeate through a solid phase extraction material that is able to selectively retain the OSI-7904 and/or the cholesterol and HSPC. A variety of supports could potentially be used that exploit either differences in lipophilicity or charge (ion exchange), or a combination of these two.

Concentration methods attempted to concentrate the OSI-7904 in aqueous solution directly included:

1) Evaporation. The water from the permeate washes could be evaporated to give the desired concentration of ˜100 mg/mL OSI-7904. The temperature used would depend on the thermal stability of the compound in aqueous solution but evaporation under vacuum could avoid high temperatures if necessary.

2) Solid phase extraction (SPE) methods. In addition to purifying the OSI-7904, SPE could be used to concentrate the drug by retaining the OSI-7904 onto an SPE support, which could then be eluted in a small volume of elution solvent.

3) Liquid-Liquid extraction. It might be possible to exploit the partitioning behavior of OSI-7904 as a function of pH to reduce the OSI-7904 solubility in water. Then the precipitated drug can be taken up into a small volume of immiscible solvent such as chloroform.

Partitioning Properties of OSI-7904 into Chloroform

The ability of OSI-7904 to partition into chloroform as a function of pH was studied using a Sirius Glpka autotitrator. Attempts were first made to determine the pKₐs of the OSI-7904 drug substance using the Sirius autotitrator, since these values are required when determining the partition coefficients by potentiometric titration. Difficulties were experienced however with the pKₐ determinations, as the compound was found to be very insoluble in aqueous media at low pHs. It is usual practice, with a compound of low solubility, to titrate the compound with various amounts of cosolvent present to determine apparent pKₐs and then perform an extrapolation back to 100% aqueous to calculate the true thermodynamic pKₐ. However, even with cosolvent (methanol) present, precipitation of the drug was still a problem and reproducible data sets were not obtained. Titration in the presence of chloroform also showed precipitation problems. This demonstrated that the solubility of OSI-7904 in chloroform was very low, even at low pH where the compound is at its most lipophilic. As a result, extraction of OSI-7904 with chloroform is not a practical proposition since large volumes of chloroform would be required to solubilise the drug.

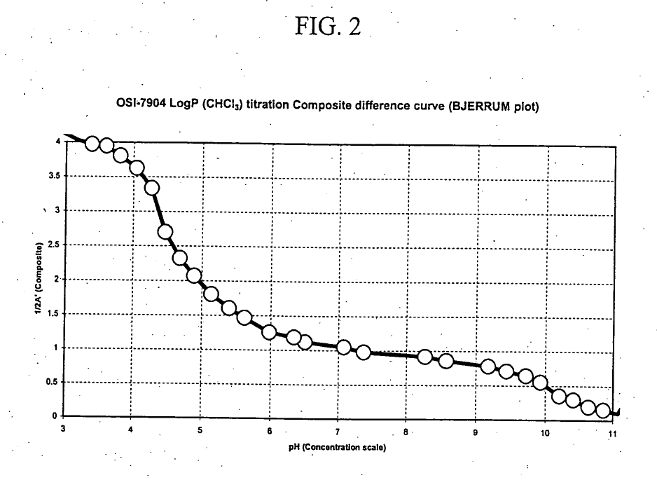

FIG. 2 shows an example of a Bjerrum plot obtained from the titration of OSI-7904 in the presence of chloroform. In this case, the Bjerrum plot is for the titration of 10 mL of 0.33 mM OSI-7904 aqueous solution in the presence of 5 mL chloroform. Titration started at low pH. The sharp change in slope at pH˜4.5 and lack of symmetry in the curve is indicative of undissolved compound present which then dissolves as the pH is increased. This plot shows that the solubility of OSI-7904 in chloroform is likely to be less than 0.4 mg/mL.

Partitioning Properties of HSPC and Cholesterol into Chloroform

Concentrations of 1 mg/mL and 0.1 mg/mL of the 2:1 molar ratio HSPC:cholesterol spray dried mix dispersions were produced. Then the mixes were extracted with chloroform and varying chloroform:aqueous ratios at 1:3, 1:1 and 3:1. It was found that a ratio of 1:3 gave the clearest definition of the two phases after equilibration. The other ratios produced milky emulsions in the aqueous phase.

HSPC consists of two main components: PSPC (1-palmitoyl-2-stearoyl-sn-glyero-3-phosphocholine) and DSPC (1,2-distearoyl-sn-glycero-3-phosphocholine). HPLC analysis of the 1 mg/mL cholesterol:HSPC dispersion after extraction with chloroform (1:3, CHCl₃:Aq volume ratio) showed that cholesterol and HSPC levels were undetectable. For the 0.1 mg/mL dispersion the extraction was less efficient; being ˜95% for cholesterol, ˜63% for PSPC and ˜24% for DSPC.

Partitioning Properties of HSPC and Cholesterol into Chloroform in the Presence of OSI-7904

A 17 mg/mL OSI-7904 solution containing ˜0.5 mg/mL, 2:1 molar ratio HSPC:cholesterol mix was produced and extracted by mechanical shaking for 1 hour with chloroform at the 1:3 (CHCl₃:Aq) ratio. Before partition the aqueous phase was hazy and yellow in color. After extraction the upper aqueous phase was clear and yellow.

The upper aqueous phase was analyzed for HSPC, cholesterol and OSI-7904. Approximately 94% of OSI-7904 was retained in the aqueous phase and approximately 2%, 8% and 8% retained of cholesterol, DSPC and PSPC respectively. This aqueous phase was extracted a second time with chloroform. The amounts retained in the aqueous phase for PSPC, DSPC and cholesterol were approximately 65%, 100% and 67% respectively for this second extraction. This suggested that the efficiency in removing the contaminants decreased with decreasing contaminant concentration. It is interesting to note also that chloroform extraction did not alter the drug related impurity profile.

Experiments with Saturated HSPC:Cholesterol Solutions

The work above had been carried out with dispersions of solid HSPC:cholesterol. It was thought that the actual permeate would only contain materials in free solution. A better model for this was considered to be saturating some water with the HSPC:cholesterol mix, filtering out the solid and working with this clarified solution. A saturated solution prepared in this way typically contained only traces of cholesterol and HSPC. It was noticed however that on one occasion when the dispersion of HSPC:cholesterol in water had been stirred continuously for approximately four days, the filtrate was opalescent and not clear and the levels of the HSPC and cholesterol were much higher. It was thought the most likely explanation for this was that initially multilamellar liposomes were formed and these were filtered out leaving only HSPC and cholesterol that are in free solution or in micelles. On extending mixing, the multilamellar liposomes are broken down to smaller particles and these are able to pass through the pores of the filter.

A 1.2 mg/mL solution of OSI-7904 was prepared containing trace levels of HSPC and cholesterol in solution (no solid present). This was extracted with chloroform in the 1:3 ratio (8.8 mL:26.5 mL, CHCl₃:Aq) by manually shaking for 1 min. It was noticed that some insoluble material was present in the sample after shaking and standing for 1 h. This may have been OSI-7904 that had precipitated at the interface. The aqueous phase was filtered through a 0.45 μm aperture filter and analyzed for OSI-7904, cholesterol and HSPC by HPLC. The trace levels of DSPC and PSPC were undetectable in the presence of OSI-7904 in samples before extraction due to the sloping baseline caused by the tail of the OSI-7904 peak. As a result, it was not possible to determine whether the level of HSPC had been reduced by extraction There was no decrease in the concentration of cholesterol in the aqueous phase after extraction with chloroform. The recovery of OSI-7904 in the aqueous phase after extraction with chloroform was approximately 94%.

Conclusions from Chloroform Extractions

When cholesterol and HSPC are present as a solid dispersion, these may be mostly removed using chloroform extractions. However, when present at trace levels (in the order of 10 μg/mL) in solution, chloroform is not effective in removing these materials.

Solid Phase Extraction Studies (SPE)

There are a number of ways in which SPE could be potentially used for purification and/or concentration. One is where the contaminants are adsorbed onto the SPE material and the OSI-7904 passes through unretained. This will purify the OSI-7904 but a concentration step will then be needed. Another way is if the OSI-7904 is retained on the SPE material and the contaminants pass through. The OSI-7904 could then be washed off the SPE material with a small volume of suitable elution solvent allowing the drug to be separated from the contaminants and concentrated into a small volume at the same time.

The solid phase extraction studies undertaken are summarized in the table below. The procedure involved firstly conditioning the SPE material with methanol and then water. A sample of the OSI-7904 in the presence of the lipid contaminants were then passed through the SPE material and the liquid that passed through (first pass) collected and analyzed for OSI-7904, cholesterol and HSPC by HPLC. If the drug was retained then efforts were made to wash the material off the solid support with an appropriate elution solvent.

| SPE phase | First pass | Elution 1 | Elution 2 |
|---|---|---|---|
| C18 (100 mg/1 mL) | No retention | N/A | N/A |
| Conditioned with 2 mL of | of cholesterol | | |
| methanol followed by 2 mL water. | | | |
| 2 mL sample added. | | | |
| Waters MAX (500 mg/6 cc) | Retention of | 6 mL 2% formic acid in | N/A |
| Conditioned with 6 mL methanol | OSI-7904, | methanol used. | |
| followed by 6 mL water. 6 mL | cholesterol | OSI-7904, cholesterol | |
| sample added. | and HSPC | HSPC eluted | |
| SAX (500 mg/6 cc) | Retention of | 1 mL 2% formic acid in | N/A |
| Conditioned with 2 mL methanol | OSI-7904 | methanol used. | |
| followed by 2 mL of water. 1 mL | | No elution of OSI-7904. | |
| sample added. | | | |
| Waters MAX (500 mg/6 mL) | Retention of | 6 mL methanol used. | 6 ml 2% formic |
| Conditioned with 6 ml methanol | most of | Most cholesterol and | acid in methanol used. |
| followed by 6 mL water. 6 mL | cholesterol | HSPC eluted. | Practically no |
| sample added. | and HSPC and | OSI-7904 retained | cholesterol: |
| | all the | but two of the | HSPC eluted. |
| | OSI-7904 | major drug related | OSI-7904 eluted. |
| | | impurities (RRT = | |
| | | 1.83 and 1.96) eluted. | |

Waters MAX is a mixed mode phase combining reversed phase character with strong anion exchange. SAX—(Radley) is a strong anion exchange resin.

Discussion of SPE Results

Surprisingly, the lipid components cholesterol and HSPC are not retained on the C18 phase.

The Waters MAX phase retained the OSI-7904, cholesterol and HSPC. The mechanism of retention for the OSI-7904 is considered to be by anion exchange. For cholesterol, the mechanism is likely to be by hydrophobic interaction between the lipophilic character of the stationary phase and the lipophilicity of the compound. Although if this is the case, it is not known why cholesterol was not retained on the C18 phase or removed by chloroform extraction—the mechanism of retention may be more complex. For the HSPC components DSPC and PSPC, these are zwitterionic and so the interaction may also be by hydrophobic interaction. The lipid components are successfully removed on elution with methanol, leaving behind the OSI-7904 on the stationary phase. Interestingly, the two major drug related impurities (RRT 1.83 and 1.96) were also eluted with methanol only. OSI-7904 was successfully eluted with 2% formic acid in methanol. It may therefore be possible to separate OSI-7904 away from HSPC, cholesterol and some drug related impurities using this type of phase. Elution with a small volume of 2% formic acid in methanol means that the methanol and formic acid would need to be removed by evaporation. This would leave behind the solid that would need to be redissolved in water containing NaOH before proceeding.

The SAX phase retained the OSI-7904 but the OSI-7904 was not eluted with the 2% formic acid in methanol. To elute the drug from this phase it might be necessary to use a high ionic strength aqueous solution (sodium chloride solution for example). Recovery of the OSI-7904 from such a solution would not be straightforward.

Work With OSI-7904L Permeate Washes

Three permeate washes from an actual OSI-7904L drug product manufacture were obtained. The three washes were i) wash 1, ii) washes 2 and 3 (pooled together) and iii) washes 5 and 6 (pooled together). The OSI-7904 concentrations in these three washes were estimated to be 9, 6 and 4 mg/mL respectively.

These solutions were clear with a yellow coloration due to the OSI-7904. The solutions were analyzed for OSI-7904, cholesterol and HSPC by HPLC. The concentrations were found to be as follows:

| | Approximate OSI-7904 | Cholesterol |
|---|---|---|
| Permeate Wash | concentration (mg/mL)* | concentration (μg/mL) |
| 1 | 6.4 | 0.31 |
| 2 and 3 | 1.9 | 0.17 |
| 4 and 5 | 0.5 | 0.08 |

*The assayed values for OSI-7904 were as mg/g but an assumption has been made for the density of the permeate wash as being 1 g/mL to obtain values in mass/volume

The levels of DSPC and PSPC in the washes were undetectable. It has been estimated that the limit of detection for DSPC and PSPC in the presence of OSI-7904 is 3 μg/mL for both compounds. The concentration of HSPC in wash 1 is therefore likely to be less than 6 μg/mL. The concentration of OSI-7904 in wash 1 was 6.4 mg/ml, and so a dilution of approximately 13 fold is required to reduce the drug concentration to that desired in the product (i.e. 0.5 mg/mL). The HSPC concentration carried forward from the recycle into the product will therefore be less than 0.5 μg/mL. This represents a level of less than 0.01% of that added into the product as part of the process. (The target level of of HSPC in the final product is 6.3 mg/mL. A similar calculation for cholesterol shows that the level of cholesterol carried forward from the recyle into the product would be around 0.02% of that added to the product. The levels for the lipids were lower than anticipated and correspond approximately to the concentrations observed when water was saturated with the HSPC:cholesterol mix and filtered. It was demonstrated that such low levels cannot be successfully removed by extraction with chloroform.

When 50 mL of permeate wash 1 was evaporated down to 4.5 mL, the solution remained essentially clear with only a very small amount of insoluble material present. This material could be easily filtered out if necessary.

Accordingly, the present invention provides a method to reclaim un-encapsulated OSI-7904 by pooling the washes and removing the excess water by evaporation until a concentration in the order of, for example, 80 mg/mL OSI-7904 is achieved. The solution can then be taken and further fresh drug and sodium hydroxide added to achieve a predetermined drug manufacturing level of, for example, 100 mg/mL and the manufacturing process continued.

Thus, the present invention provides a method of forming a liposomal pharmaceutical composition containing the compound: 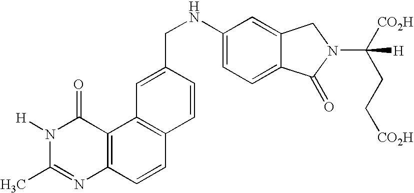
from a feedstock solution of the compound, comprising the steps of

assaying a permeate wash for HSPC and cholesterol;

processing the permeate wash to form a concentrated solution of the compound if the assay result is lower or equal to a predetermined value; and

adding the concentrated solution to the feedstock solution.

The permeate wash can be formed from the pooling of a multitude or plurality of permeate washes. The permeate washes can be from any of a series of washes. The assaying can include assaying for the compound itself. The processing can be evaporating the water. There can be the steps of assaying the concentration of the compound in the concentrated solution and adjusting the concentration as needed by adding water or further processing to remove water.

Further, the method includes extracting the permeate wash to form an extracted solution if the assay result is higher than a predetermined value. Such extraction can form a waste liquid that can be disposed of. The method includes testing the extracted solution, to form a tested solution, to determine if the HSPC and cholesterol is higher than a predetermined value. The method also includes processing the tested solution to form a concentrated tested solution of the compound if the test results are not higher than a predetermined value. The method also includes adding the concentrated tested solution to the feedstock solution. The method also includes steps of removing the extracting liquid from the extracted solution, quantifying the extracted solution for extracting liquid to form a quantified extracted solution, returning the quantified extracted solution to the extracted liquid for further removal of extracting liquid if the quantification is too high, and using the quantified extracted solution in the processing the tested solution step if the quantification is sufficiently low in extracted liquid.

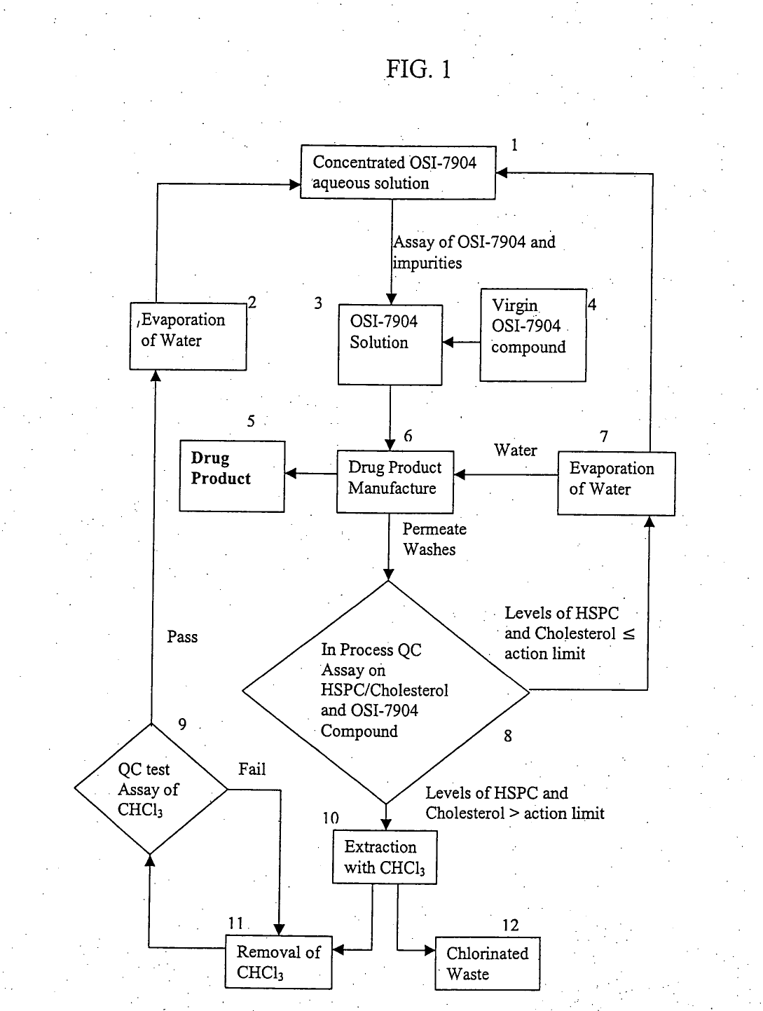

A schematic for this process is shown in FIG. 1. Referring to FIG. 1, the drug manufacturing process for liposomal OSI-7904 includes the introduction of virgin OSI-7904 compound (box 4) to form an OSI-7904 solution (feedstock) at a predetermined drug manufacturing concentration level (box 3). The solution is processed in the drug manufacturing process (box 6) to produce the liposomal drug product (box 5).

As a byproduct of the drug product manufacturing process (box 6), permeate washes are produced, which would be assayed (box 8) for HSPC/cholesterol and optionally for OSI-7904. If the assay determines that the levels of HSPC and cholesterol are above a predetermined action limit, the wash is extracted with CHCl₃ (box 10). If the assay determines that the levels of HSPC and cholesterol are equal or below a predetermined action limit, the wash is concentrated by evaporating off water (box 7).

The water evaporated from the evaporating step (box 7) can be sent to the drug manufacturing process (box 6). The concentrated OSI-7904 solution (box 1) is assayed for OSI-7904 and impurities. If passed, the solution is adjusted to the predetermined drug manufacturing concentration level (box 3) and used in the drug product manufacturing process (box 6)—thus, reclaiming the OSI-7904 from the permeate washes.

The extraction process (box 10) will lead to chlorinated waste (box 12) that is disposed of appropriately. The non-waste has CHCl₃ removed (box 11). The resulting mixture is assayed for CHCl₃ (box 9) and, if failing, sent back for further removal of CHCl₃. If passing, the resulting mixture is concentrated by evaporation of water (box 2) and the concentrated OSI-7904 solution (box 1) is assayed as described above and the OSI-7904 reclaimed for use.

What is claimed is:

1. A method of formation of a liposomal pharmaceutical composition containing the compound: 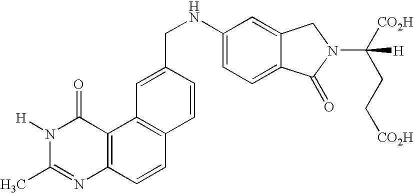
from a feedstock solution of the compound, comprising the steps of:
assaying a permeate wash for HSPC and cholesterol;
processing the permeate wash, to form a concentrated solution of the compound, if the assay result is lower or equal to a predetermined value; and
adding the concentrated solution to the feedstock solution.

2. The method according to claim 1, further including the steps of:
extracting the permeate wash, to form an extracted wash, if the assay result is higher to a predetermined value;
reassaying the extracted wash for HSPC and cholesterol;
processing the extracted wash, to form a second concentrated solution of the compound, if the reassay result is lower or equal to a predetermined value; and
adding the second concentrated solution to the feedstock solution.

3. The method according to claim 1, further including the step of:
utilizing the clean liquid, resulting from the processing step, in the formation of the pharmaceutical formulation.

4. The method according to claim 2, further including the step of:
utilizing the clean liquid, resulting from the processing the extracted wash step, in the formation of the pharmaceutical formulation.
